(19) United States
(12) Patent Application Publication
(10) Pub. No.: US 2011/0319482 A1
(43) Pub. Date: Dec. 29, 2011

(54) NOVEL TREATMENTS

(76) Inventors: Peter Blower, Essex (GB); Paul Sharpe, Essex (GB); Jes Oleson, Glostrup (DK); Andrew Parsons, Essex (GB)

(21) Appl. No.: 12/737,065

(22) PCT Filed: Jun. 4, 2009
(86) PCT No.: PCT/GB2009/050624

§ 371 Date: Sep. 19, 2011

Related U.S. Application Data
(60) Provisional application No. 61/105,204, filed on Oct. 14, 2008.

(30) Foreign Application Priority Data
Jun. 5, 2008 (GB) 0810302.0
Oct. 10, 2008 (GB) 0818628.0

(51) Int. Cl. A61K 31/353; A61P 11/00; A61P 25/24; A61P 21/00; A61P 3/04; A61P 25/00; A61P 9/00; A61P 9/10; A61P 25/30; A61P 31/00; A61P 3/08; A61P 35/00; A61P 25/06; A61P 25/08

(52) U.S. Cl. 514/456

(57) ABSTRACT

The present invention relates to the treatment of the premonitory symptoms of migraine, to the treatment of aura associated with or without migraine, epilepsy, non-epileptic seizures, stroke or other cardiovascular disorders, to the pre-emptive treatment of aura, migraine, epilepsy, stroke or other cardiovascular disorders, to the treatment of migraine recurrence or aura recurrence, and to tonabersat, co-crystals thereof, and compositions comprising tonabersat for use in said treatments.

The present invention relates to the treatment of the premonitory symptoms of migraine, to the treatment of aura associated with or without migraine, epilepsy, non-epileptic seizures, stroke or other cardiovascular disorders, to the pre-emptive treatment of aura, migraine, epilepsy, stroke or other cardiovascular disorders, to the treatment of migraine recurrence or aura recurrence, and to tonabersat or an analogue of formula 1, co-crystals of tonabersat, and compositions comprising tonabersat or an analogue of formula 1 for use in said treatments.

International patent application WO 95/34545 discloses a series of specific named compounds, including tonabersat, which possess anticonvulsant activity and are said to be useful in the treatment or prevention of CNS disorders, including, inter alia, migraine, epilepsy and cerebral ischemia. Tonabersat, otherwise known as cis-6-acetyl-4-(S)-(3-chloro-4-fluorobenzoylamino)-3,4-dihydro-2,2-dimethyl-2H-1-benzopyran-3-(S)-ol, is a member of the class of drugs called neuronal gap junction blockers, which is currently being investigated for a range of conditions including migraine, epilepsy, pain and other neurological conditions.

U.S. Pat. No. 5,948,811 (incorporated herein by way of reference) describes a class of compounds (‘the analogues of formula I’) which may be used for the prophylaxis and treatment of disorders within the central and peripheral nervous system, including migraine with or without aura.

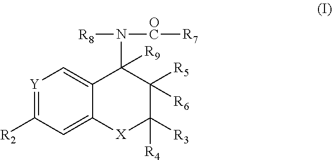

Y is C—R₁;
R₁ is acetyl;
R₂ is hydrogen, C₃₋₈ cycloalkyl, C₁₋₆ alkyl optionally interrupted by oxygen or substituted by hydroxy, C₁₋₆ alkoxy or substituted aminocarbonyl, C₁₋₆alkylcarbonyl, C₁₋₆ alkoxycarbonyl, C₁₋₆ alkylcarbonyloxy, C₁₋₆ alkoxy, nitro, cyano, halo, trifluoromethyl, or CF₃S; or a group CF₃-A-, where A is —CF₂—,
—CO—, —CH₂—, CH(OH), SO₂, SO, CH₂—O, or CONH; or a group CF₂ H-A′- where A′ is oxygen, sulphur, SO, SO₂, CF₂ or CFH; trifluoromethoxy, C₁₋₆ alkylsulphinyl, perfluoro C₂₋₆ alkylsulphonyl, C₁₋₆ alkylsulphonyl, C₁₋₆ alkoxysulphinyl, C₁₋₆ alkoxysulphonyl, aryl, heteroaryl, arylcarbonyl, heteroarylcarbonyl, phosphono, arylcarbonyloxy, heteroarylcarbonyloxy, arylsulphinyl, heteroarylsulphinyl, arylsulphonyl, or heteroarylsulphonyl in which any aromatic moiety is optionally substituted, C₁₋₆ alkylcarbonylamino, C₁₋₆ alkoxycarbonylamino, C₁₋₆ alkyl-thiocarbonyl, C₁₋₆ alkoxy-thiocarbonyl, C₁₋₆ alkyl-thiocarbonyloxy, 1-mercapto C₂₋₇ alkyl, formyl, or aminosulphinyl, aminosulphonyl or aminocarbonyl, in which any amino moiety is optionally substituted by one or two C₁₋₆ alkyl groups, or C₁₋₆ alkylsulphinylamino, C₁₋₆ alkylsulphonylamino, C₁₋₆ alkoxysulphinylamino or C₁₋₆ alkoxysulphonylamino, or ethylenyl terminally substituted by C₁₋₆ alkylcarbonyl, nitro or cyano, or —C(C₁₋₆ alkyl)NOH or —C(C₁₋₆ alkyl)NNH₂; or amino optionally substituted by one or two C₁₋₆ alkyl or by C₂₋₇ alkanoyl; one of R₃ and R₄ is hydrogen or C₁₋₄ alkyl and the other is C₁₋₄ alkyl, CF₃ or CH₂X^{a} is fluoro, chloro, bromo, iodo, C₁₋₄ alkoxy, hydroxy, C₁₋₄ alkylcarbonyloxy, —S—C₁₋₄ alkyl, nitro, amino optionally substituted by one or two C₁₋₄ alkyl groups, cyano or C₁₋₄ alkoxycarbonyl; or R₃ and R₄ together are C₂₋₅ polymethylene optionally substituted by C₁₋₄ alkyl;
R₅ is C₁₋₆ alkylcarbonyloxy, benzoyloxy, ONO₂, benzyloxy, phenyloxy or C₁₋₆ alkoxy and R₆ and R₉ are hydrogen or R₅ is hydroxy and R₆ is hydrogen or C₁₋₂ alkyl and R₉ is hydrogen;
R₇ is heteroaryl or phenyl, both of which are optionally substituted one or more times independently with a group or atom selected from chloro, fluoro, bromo, iodo, nitro, amino optionally substituted once or twice by C₁₋₄ alkyl, cyano, azido, C₁₋₄ alkoxy, trifluoromethoxy and trifluoromethyl;
R₈ is hydrogen, C₁₋₆ alkyl, OR₁₁ or NHCOR₁₀ wherein R₁₁ is hydrogen, C₁₋₆ alkyl, formyl, C₁₋₆ alkanoyl, aroyl or aryl-C₁₋₆ alkyl and R₁₀ is hydrogen, C₁₋₆ alkyl, C₁₋₆ alkoxy, mono or di C.sub.1-6 alkyl amino, amino, amino-C.sub.1-6 alkyl, hydroxy-C₁₋₆ alkyl, halo-C₁₋₆ alkyl, C₁₋₆ acyloxy-C₁₋₆ alkyl, C₁₋₆ alkoxycarbonyl-C₁₋₆-alkyl, aryl or heteroaryl; the R₈—N—CO—R₇ group being cis to the R₅ group; and X is oxygen or NR₁₂ where R₁₂ is hydrogen or C₁₋₆ alkyl.

Clinical trials (US Government Identifier NCT00311662) relate to the prophylaxis and treatment of migraine disorders including migraine with aura and migraine without aura by administration of tonabersat. International patent application WO 00/66115 refers to pre-emptive prophylactic treatment of the headache phase of migraine via administration of 5-HT₁ receptor agonists. International patent application WO 00/06161 refers to prevention of migraine recurrence via administration of the 5-HT₁ receptor agonist, eletriptan.

Migraine is a common disorder and can be divided into two major sub-types. Migraine without aura is a clinical syndrome characterised by headache with specific features and associated symptoms. Migraine with aura is primarily characterised by the focal neurological symptoms that usually precede or sometimes accompany the headache.

Another, independent, phase that may be experienced by some patients is a premonitory phase, which can occur hours or days before the headache. The International Headache Classification Society define premonitory symptoms as symptoms preceding and forewarning of a migraine attack by 2-48 hours, occurring before the aura in migraine with aura and before the onset of pain in migraine without aura (Cephalalgia, 1988, 8, Supp. 7, 1-96). The premonitory symptoms may include excitory and/or inhibitory symptoms.

Among the common premonitory symptoms are irritability, euphoria, elation, physical hyperactivity, fatigue, excessive yawning, excessive sleepiness, increased sensitivity to light and sound, unusual hunger, craving for certain foods, depression, mental withdrawal, behaviour sluggishness, feeling tired, poor concentration, muscle weakness, anorexia and fluid retention (Cephalalgia, 1988, 8, Supp. 7, 1-96, Kelman, L., The Premonitory Symptoms (Prodrome): A Tertiary Care Study of 893 Migraineurs, Headache, 2004, 44(9),865-872).

A distinction must be made between the prophylactic treatment of migraine wherein a drug is continually administered to a patient in order to prevent the occurrence of a migraine attack, and the pre-emptive treatment of migraine via administration during the pre-headache phase, during either the premonitory symptom phase or the aura phase. The premonitory symptoms and/or aura may be experienced by patients with or without an associated migraine headache and therefore require treatment in their own right.

Further distinctions must be made between the treatment of migraine, which involves the treatment of an established migraine headache, treatment of migraine recurrence, which involves treatment of an established migraine headache recurrence, and the prevention of migraine recurrence, which involves treating a patient in anticipation of a migraine headache recurrence in order to prevent that recurrence. Migraine recurrence is defined as the return of a moderate or severe migraine headache within 24 hours of the first dosing with medication, from a state of no, or mild, migraine headache within 2 hours of the first dosing with medication.

It is believed that migraine is a risk factor for stroke (Jousilahti, P. et. al. Headache and the Risk of Stroke, Archives of Internal Medicine, 163(9), 1058-1062), and patients who suffer from migraine with aura have been shown to be of greater risk from stroke than those who suffer from migraine without aura (Kurth T. et. al., Migraine, vascular risk, and cardiovascular events in women: prospective cohort study, British Medical Journal, 16 Aug. 2008, 337-383). The treatment of aura may therefore prove beneficial in the prevention of stroke.

Migraine with aura has also been associated with increased risk of other major cardiovascular disease events, such as myocardial infarction, coronary revascularisation and angina (Kurth T. et. al., Migraine and Risk of Cardiovascular Disease in Women, JAMA, 2006, 296(3), 283-291). The treatment of aura may therefore prove beneficial in the prevention of these and other related diseases.

The research suggests a greater link between migraine with aura in women and the above-mentioned diseases and therefore treatment of this group would be of particular benefit.

Aura is also experienced by many epileptics in advance of tonic clonic seizures and the aura itself is classed as a simple partial seizure. The aura may be experienced hours or days prior to the tonic clonic seizure and this forewarning may enable action to be taken to prevent or limit the effect of the main seizure. The treatment of aura may therefore prove beneficial in the treatment of epilepsy and the prevention of seizures.

Aura may also by experienced in advance non-epileptic seizures. This forewarning may enable action to be taken to prevent or limit the effect of the seizure. The treatment of aura may therefore prove beneficial in the treatment or prevention of non-epileptic seizures.

There exists a need for safe, alternative and/or improved methods and compositions for the treatment of migraine and of the various symptoms and conditions associated with migraine. In particular there remains a need for alternative and/or improved methods and compositions for the treatment of the premonitory symptoms that may signal the onset of a migraine attack and the pre-emptive treatment of migraine via administration of an effective drug during the premonitory symptom phase. There also exists a need for alternative and/or improved methods and compositions for the treatment or prevention of migraine recurrence.

Additionally, there is a need for alternative and/or improved methods and compositions for the treatment of aura that may be associated with, or independent from, a migraine headache. Such methods and compositions may have benefit in the treatment or prevention of migraine, epilepsy, non-epileptic seizures, stroke, or major cardiovascular disease events, such as myocardial infarction, coronary revascularisation and angina. Alternatively, such methods and compositions may reduce the severity of any event subsequent to the aura phase in such diseases. The treatment of such aura may be acute or prophylactic.

There is also a need for alternative and/or improved methods and compositions for the pre-emptive treatment of epilepsy, non-epileptic seizures, stroke and cardiovascular diseases. Pre-emptive treatment would, for instance, involve administration of a drug prior to any epileptic fit, stroke, or cardiovascular infarction, during a phase where symptoms signalling any such event are experienced. In particular, administration of a drug during the aura phase that may precede any such potential event would be beneficial.

For acute treatment, a rapid onset of action is preferred, and therefore, drugs that reach maximum plasma concentrations shortly after administration would be most beneficial. Accordingly, compositions providing rapid drug-release and/or dissolution are preferred.

It has now been surprisingly found that tonabersat and analogues of formula I

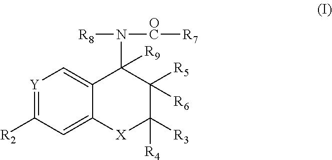

Y is C—R₁;
R₁ is acetyl;
R₂ is hydrogen, C₃₋₈ cycloalkyl, C₁₋₆ alkyl optionally interrupted by oxygen or substituted by hydroxy, C₁₋₆ alkoxy or substituted aminocarbonyl, C₁₋₆ alkylcarbonyl, C₁₋₆ alkoxycarbonyl, C₁₋₆ alkylcarbonyloxy, C₁₋₆ alkoxy, nitro, cyano, halo, trifluoromethyl, or CF₃S; or a group CF₃-A-, where A is —CF₂—, —CO—, —CH₂—, CH(OH), SO₂, SO, CH₂—O, or CONH; or a group CF₂H-A′- where A′ is oxygen, sulphur, SO, SO₂, CF₂ or CFH; trifluoromethoxy, C₁₋₆ alkylsulphinyl, perfluoro C₂₋₆ alkylsulphonyl, C₁₋₆ alkylsulphonyl, C₁₋₆ alkoxysulphinyl, C₁₋₆ alkoxysulphonyl, aryl, heteroaryl, arylcarbonyl, heteroarylcarbonyl, phosphono, arylcarbonyloxy, heteroarylcarbonyloxy, arylsulphinyl, heteroarylsulphinyl, arylsulphonyl, or heteroarylsulphonyl in which any aromatic moiety is optionally substituted, C₁₋₆ alkylcarbonylamino, C₁₋₆ alkoxycarbonylamino, C₁₋₆ alkyl-thiocarbonyl, C₁₋₆ alkoxy-thiocarbonyl, C₁₋₆ alkyl-thiocarbonyloxy, 1-mercapto C₂₋₇ alkyl, formyl, or aminosulphinyl, aminosulphonyl or aminocarbonyl, in which any amino moiety is optionally substituted by one or two C₁₋₆ alkyl groups, or C₁₋₆ alkylsulphinylamino, C₁₋₆ alkylsulphonylamino, C₁₋₆ alkoxysulphinylamino or C₁₋₆ alkoxysulphonylamino, or ethylenyl terminally substituted by C₁₋₆ alkylcarbonyl, nitro or cyano, or —C(C₁₋₆ alkyl)NOH or —C(C₁₋₆ alkyl)NNH₂; or amino optionally substituted by one or two C₁₋₆ alkyl or by C₂₋₇ alkanoyl; one of R₃ and R₄ is hydrogen or C₁₋₄ alkyl and the other is C₁₋₄ alkyl, CF₃ or CH₂ X^{a} is fluoro, chloro, bromo, iodo, C₁₋₄ alkoxy, hydroxy, C₁₋₄ alkylcarbonyloxy, —S—C₁₋₄ alkyl, nitro, amino optionally substituted by one or two C₁₋₄ alkyl groups, cyano or C₁₋₄ alkoxycarbonyl; or R₃ and R₄ together are C₂₋₅ polymethylene optionally substituted by C₁₋₄ alkyl;
R₅ is C₁₋₆ alkylcarbonyloxy, benzoyloxy, ONO₂, benzyloxy, phenyloxy or C₁₋₆ alkoxy and R₆ and R₉ are hydrogen or R₅ is hydroxy and R₆ is hydrogen or C₁₋₂ alkyl and R₉ is hydrogen;
R₇ is heteroaryl or phenyl, both of which are optionally substituted one or more times independently with a group or atom selected from chloro, fluoro, bromo, iodo, nitro, amino optionally substituted once or twice by C₁₋₄ alkyl, cyano, azido, C₁₋₄ alkoxy, trifluoromethoxy and trifluoromethyl;
R₈ is hydrogen, C₁₋₆ alkyl, OR₁₁ or NHCOR₁₀ wherein R₁₁ is hydrogen, C₁₋₆ alkyl, formyl, C₁₋₆ alkanoyl, aroyl or aryl-C₁₋₆ alkyl and R₁₀ is hydrogen, C₁₋₆ alkyl, C₁₋₆ alkoxy, mono or di C.sub.1-6 alkyl amino, amino, amino-C.sub.1-6 alkyl, hydroxy-C₁₋₆ alkyl, halo-C₁₋₆ alkyl, C₁₋₆ acyloxy-C₁₋₆ alkyl, C₁₋₆ alkoxycarbonyl-C₁₋₆-alkyl, aryl or heteroaryl; the R₈—N—CO—R₇ group being cis to the R₅ group; and X is oxygen or NR₁₂ where R₁₂ is hydrogen or C₁₋₆ alkyl, are capable of treating aura in patients suffering from migraine with aura.

A preferred analogue of formula 1 is the compound carabersat or (trans-(+)-6-acetyl-4-(S)-(4-fluorobenzoylamino)-3,4-dihydro-2,2-dimethyl-2H-1-benzo[b]pyran-3R-ol,hemihydrate.

For therapeutic administration according to the present invention, tonabersat or an analogue of formula I, is most preferably employed in the form of its free base, but may also be used in the form of a pharmaceutically acceptable salt, preferably the hydrochloride salt. Alternative salts with pharmaceutically acceptable acids may also be utilised in prophylactic and/or therapeutic administration, for example salts derived from acids including, but not limited to, hydrobromic acid, phosphoric acid, acetic acid, fumaric acid, maleic acid, salicylic acid, citric acid, oxalic acid, lactic acid, malic acid, methanesulphonic acid and p-toluene sulphonic acid.

All references to tonabersat or an analogue of formula I, herein includes all pharmaceutically acceptable salts, and all solvates thereof.

Additionally, tonabersat and analogues of formula I have been found to provide persistent or carry-over benefits in the treatment of aura and migraine once treatment has ceased. Potential benefits include provision for prophylactic treatment regimes with periods of non-administration.

In a first aspect, the present invention provides for tonabersat or an analogue of formula I, or a pharmaceutically acceptable composition thereof, for use in the treatment of one or more of the premonitory symptoms of migraine. The migraine may be classed as migraine with aura or migraine without aura. The treatment of such symptoms may be acute or prophylactic.

In a further aspect, the present invention provides for the use of tonabersat or an analogue of formula I, or a pharmaceutically acceptable composition thereof, in the manufacture of a medicament for the treatment of one or more of the premonitory symptoms of migraine or any other symptom or disorder listed below.

Accordingly, the present invention provides a method for the treatment of one or more of the premonitory symptoms of migraine comprising administering to a patient in need thereof a pharmaceutically effective amount of tonabersat or an analogue of formula I, or a pharmaceutically acceptable composition thereof.

In particular, the present invention provides a method for the treatment of one or more excitory and/or inhibitory symptoms that are associated with the premonitory phase of a migraine attack, comprising administering to a patient in need thereof a pharmaceutically effective amount of tonabersat or an analogue of formula I, or a pharmaceutically acceptable composition thereof.

More specifically, the present invention provides a method for the treatment of one or more of the symptoms of irritability, euphoria, elation, physical hyperactivity, fatigue, excessive yawning, excessive sleepiness, rhinitis, chronic rhinitis, sinusitis, increased sensitivity to light and sound, unusual hunger, craving for certain foods, depression, mental withdrawal, behaviour sluggishness, feeling tired, poor concentration, muscle weakness, anorexia and fluid retention, that are associated with the premonitory phase of a migraine attack, comprising administering to a patient in need thereof a pharmaceutically effective amount of tonabersat or an analogue of formula I, or a pharmaceutically acceptable composition thereof

In another aspect, the present invention provides for tonabersat or an analogue of formula I, or a pharmaceutically acceptable composition thereof, for use in the treatment of one or more of the premonitory symptoms of migraine. The treatment of such symptoms may be acute or prophylactic.

In particular, the present invention provides for tonabersat or an analogue of formula I, or a pharmaceutically acceptable composition thereof, for use in the treatment of one or more excitory and/or inhibitory symptoms that are associated with the premonitory phase of a migraine attack.

More specifically, the present invention provides for tonabersat or an analogue of formula I, or a pharmaceutically acceptable composition thereof for use in the treatment of one or more of the symptoms of irritability, euphoria, elation, physical hyperactivity, fatigue, excessive yawning, excessive sleepiness, rhinitis, chronic rhinitis, sinusitis, increased sensitivity to light and sound, unusual hunger, craving for certain foods, depression, mental withdrawal, behaviour sluggishness, feeling tired, poor concentration, muscle weakness, anorexia and fluid retention, that are associated with the premonitory phase of a migraine attack.

In a further aspect, the present invention provides for the use of tonabersat or an analogue of formula I, or a pharmaceutically acceptable composition thereof, for the pre-emptive treatment of migraine. The migraine may be classed as migraine with aura or migraine without aura.

Accordingly, the present invention provides a method for the pre-emptive treatment of migraine comprising administering to a patient in need thereof a pharmaceutically effective amount of tonabersat or an analogue of formula I, or a pharmaceutically acceptable composition thereof, during the premonitory symptom phase associated with a migraine attack.

In particular, the present invention provides a method for the pre-emptive treatment of migraine comprising administering to a patient in need thereof a pharmaceutically effective amount of tonabersat or an analogue of formula I, or a pharmaceutically acceptable composition thereof, during the phase where one or more excitory and/or inhibitory symptoms, that are associated with the premonitory phase of a migraine attack are experienced.

More specifically, the present invention provides a method for the pre-emptive treatment of migraine comprising administering to a patient in need thereof a pharmaceutically effective amount of tonabersat or an analogue of formula I, or a pharmaceutically acceptable composition thereof, during the phase where one or more of the symptoms of irritability, euphoria, elation, physical hyperactivity, fatigue, excessive yawning, excessive sleepiness, rhinitis, chronic rhinitis, sinusitis, increased sensitivity to light and sound, unusual hunger, craving for certain foods, depression, mental withdrawal, behaviour sluggishness, feeling tired, poor concentration, muscle weakness, anorexia and fluid retention, that are associated with the premonitory phase of a migraine attack are experienced.

In another aspect, the present invention provides for tonabersat or an analogue of formula I, or a pharmaceutically acceptable composition thereof, for use in the pre-emptive treatment of migraine.

Accordingly, the present invention provides for tonabersat or an analogue of formula I, or a pharmaceutically acceptable composition thereof, for use in the pre-emptive treatment of migraine via administration during the premonitory symptom phase associated with a migraine attack.

In particular, the present invention provides for tonabersat or an analogue of formula I, or a pharmaceutically acceptable composition thereof, for use in the pre-emptive treatment of migraine via administration during the phase where one or more excitory and/or inhibitory symptoms, that are associated with the premonitory phase of a migraine attack are experienced.

More specifically, the present invention provides for tonabersat or an analogue of formula I, or a pharmaceutically acceptable composition thereof, for use in the pre-emptive treatment of migraine via administration during the phase where one or more of the symptoms of irritability, euphoria, elation, physical hyperactivity, fatigue, excessive yawning, excessive sleepiness, rhinitis, chronic rhinitis, sinusitis, increased sensitivity to light and sound, unusual hunger, craving for certain foods, depression, mental withdrawal, behaviour sluggishness, feeling tired, poor concentration, muscle weakness, anorexia and fluid retention, that are associated with the premonitory phase of a migraine attack are experienced.

In a further aspect, the present invention provides for the use of tonabersat or an analogue of formula I, or a pharmaceutically acceptable composition thereof, for the treatment or prevention of migraine recurrence. The migraine may be classed as migraine with aura or migraine without aura.

Accordingly, the present invention provides a method for the treatment or prevention of migraine recurrence comprising administering to a patient in need thereof a pharmaceutically effective amount of tonabersat or an analogue of formula I, or a pharmaceutically acceptable composition thereof.

In another aspect, the present invention provides for tonabersat or an analogue of formula I, or a pharmaceutically acceptable composition thereof, for use in the treatment or prevention of migraine recurrence.

In a further aspect, the present invention provides for the use of tonabersat or an analogue of formula I, or a pharmaceutically acceptable composition thereof, for the treatment or prevention of aura. The aura may, for example, be associated with migraine, epilepsy, non-epileptic seizures, stroke, or major cardiovascular disease events, such as myocardial infarction, coronary revascularisation or angina. The treatment of such aura may be acute or prophylactic. The patient may be either male or female.

Accordingly, the present invention provides a method for the treatment or prevention of aura comprising administering to a patient in need thereof a pharmaceutically effective amount of tonabersat or an analogue of formula I,or a pharmaceutically acceptable composition thereof.

In one embodiment, the present invention provides a method for the treatment or prevention of aura in a patient with either a history of, or at higher risk of suffering from, migraine, epilepsy, non-epileptic seizures, stroke or cardiovascular disease, including major cardiovascular disease events, such as myocardial infarction, coronary revascularisation or angina.

In another embodiment, the present invention provides a method for the treatment or prevention of aura in a patient with a history of migraine with or without aura, and preferably migraine with aura.

Additionally, the present invention provides a method for the treatment or prevention of aura comprising administering to a patient in need thereof a pharmaceutically effective amount of tonabersat or an analogue of formula I,or a pharmaceutically acceptable composition thereof, during the premonitory symptom phase associated with a migraine attack

In particular, the present invention provides a method for the treatment or prevention of aura comprising administering to a patient in need thereof a pharmaceutically effective amount of tonabersat or an analogue of formula I,or a pharmaceutically acceptable composition thereof, during the phase where one or more excitory and/or inhibitory symptoms, that are associated with the premonitory phase of a migraine attack are experienced.

More specifically, the present invention provides a method for the treatment or prevention of aura comprising administering to a patient in need thereof a pharmaceutically effective amount of tonabersat or an analogue of formula I, or a pharmaceutically acceptable composition thereof, during the phase where one or more of the symptoms of irritability, euphoria, elation, physical hyperactivity, fatigue, excessive yawning, excessive sleepiness, rhinitis, chronic rhinitis, sinusitis, increased sensitivity to light and sound, unusual hunger, craving for certain foods, depression, mental withdrawal, behaviour sluggishness, feeling tired, poor concentration, muscle weakness, anorexia and fluid retention, that are associated with the premonitory phase of a migraine attack are experienced.

In yet another embodiment, the present invention provides a method for the treatment or prevention of aura in a patient with a history of epilepsy.

In yet another embodiment, the present invention provides a method for the treatment or prevention of aura in a patient with a history of non-epileptic seizures.

Preferably, the present invention provides a method for the treatment or prevention of aura in a patient with a history of non-epileptic seizures wherein the seizures are either organic or psychogenic seizures.

More preferably, the present invention provides a method for the treatment or prevention of aura in a patient with a history of non-epileptic seizures wherein the seizures are associated with arteriovenous malformation, head injury, drug intoxication, drug toxicity, such as with aminophylline and local anaesthetics, drug withdrawal, infection, such as with meningitis and encephalitis, fever, metabolic disturbances, such as hypoglycaemia, hyponatremia and hypoxia, brain lesions, such as tumours and abscesses, eclampsia, binaural beat brainwave entrainment, haemorrhagic stroke, cerebral venous sinus thrombosis, multiple sclerosis, photophobia, or posttraumatic stress disorder.

In yet another embodiment, the present invention provides a method for the treatment or prevention of aura in a patient with a history of, or at higher risk of suffering from, a stroke, major cardiovascular disease events, such as myocardial infarction, coronary revascularisation or angina.

In another aspect, the present invention provides for tonabersat or an analogue of formula I, or a pharmaceutically acceptable composition thereof, for use in the treatment or prevention of aura. The aura may, for example, be associated with migraine, epilepsy, non-epileptic seizures, stroke, or major cardiovascular disease events, such as myocardial infarction, coronary revascularisation or angina. The treatment of such aura may be acute or prophylactic. The patient may be either male or female.

In one embodiment, the present invention provides for tonabersat or an analogue of formula I, or a pharmaceutically acceptable composition thereof, for use in the treatment or prevention of aura in a patient with either a history of, or at higher risk of suffering from, migraine, epilepsy, non-epileptic seizures, stroke, or major cardiovascular disease events, such as myocardial infarction, coronary revascularisation or angina.

In a further embodiment, the present invention provides for tonabersat or an analogue of formula I, or a pharmaceutically acceptable composition thereof, for use in the treatment or prevention of aura in a patient with a history of migraine with or without aura, and preferably migraine with aura.

Additionally, the present invention provides for tonabersat or an analogue of formula I, or a pharmaceutically acceptable composition thereof, for use in the treatment or prevention of aura via administration during the premonitory symptom phase associated with a migraine attack.

In particular, the present invention provides for tonabersat or an analogue of formula I, or a pharmaceutically acceptable composition thereof, for use in the treatment or prevention of aura via administration during the phase where one or more excitory and/or inhibitory symptoms, that are associated with the premonitory phase of a migraine attack are experienced.

More specifically, the present invention provides for tonabersat or an analogue of formula I, or a pharmaceutically acceptable composition thereof, for use in the treatment or prevention of aura via administration during the phase where one or more of the symptoms of irritability, euphoria, elation, physical hyperactivity, fatigue, excessive yawning, excessive sleepiness, rhinitis, chronic rhinitis, sinusitis, increased sensitivity to light and sound, unusual hunger, craving for certain foods, depression, mental withdrawal, behaviour sluggishness, feeling tired, poor concentration, muscle weakness, anorexia and fluid retention, that are associated with the premonitory phase of a migraine attack are experienced.

In another embodiment, the present invention provides for tonabersat or an analogue of formula I,or a pharmaceutically acceptable composition thereof, for use in the treatment or prevention of aura in a patient with a history of epilepsy.

In yet another embodiment, the present invention provides for tonabersat or an analogue of formula I,or a pharmaceutically acceptable composition thereof, for use in the treatment or prevention of aura in a patient with a history of non-epileptic seizures.

Preferably, the present invention provides for tonabersat or an analogue of formula I,or a pharmaceutically acceptable composition thereof, for use in the treatment or prevention of aura in a patient with a history of non-epileptic seizures, wherein the seizures are either organic or psychogenic seizures.

More preferably, the present invention provides for tonabersat or an analogue of formula I,or a pharmaceutically acceptable composition thereof, for use in the treatment or prevention of aura in a patient with a history of non-epileptic seizures, wherein the seizures are associated with arteriovenous malformation, head injury, drug intoxication, drug toxicity, such as with aminophylline and local anaesthetics, drug withdrawal, infection, such as with meningitis and encephalitis, fever, metabolic disturbances, such as hypoglycaemia, hyponatremia and hypoxia, brain lesions, such as tumours and abscesses, eclampsia, binaural beat brainwave entrainment, haemorrhagic stroke, cerebral venous sinus thrombosis, multiple sclerosis, photophobia, or posttraumatic stress disorder.

In yet another embodiment, the present invention provides for tonabersat or an analogue of formula I,or a pharmaceutically acceptable composition thereof, for use in the treatment or prevention of aura in a patient with a history of, or at higher risk of suffering from, a stroke, major cardiovascular disease events, such as myocardial infarction, coronary revascularisation or angina, and preferably a stroke.

In a further aspect, the present invention provides for the use of tonabersat or an analogue of formula I,or a pharmaceutically acceptable composition thereof, in the pre-emotive treatment of stroke. The patient may be either male or female and most preferably the patient is female.

Accordingly, the present invention provides a method for the pre-emptive treatment of stroke comprising administering to a patient in need thereof a pharmaceutically effective amount of tonabersat or an analogue of formula I, or a pharmaceutically acceptable composition thereof, during the aura phase associated with a potential stroke.

In another aspect, the present invention provides for tonabersat or an analogue of formula I, or a pharmaceutically acceptable composition thereof, for use in the pre-emptive treatment of stroke. The patient may be either male or female and most preferably the patient is female.

Accordingly, the present invention additionally provides for tonabersat or an analogue of formula I,or a pharmaceutically acceptable composition thereof, for use in the pre-emptive treatment of stroke via administration during the aura phase associated with a potential stroke.

In a further aspect, the present invention provides for the use of tonabersat or an analogue of formula I, or a pharmaceutically acceptable composition thereof, in the pre-emptive treatment of major cardiovascular disease events, such as myocardial infarction, coronary revascularisation and angina. The patient may be either male or female and most preferably the patient is female.

Accordingly, the present invention provides a method for the pre-emptive treatment of major cardiovascular disease events, such as myocardial infarction, coronary revascularisation and angina, comprising administering to a patient in need thereof a pharmaceutically effective amount of tonabersat or an analogue of formula I, or a pharmaceutically acceptable composition thereof, during the aura phase associated with the potential disease-related event.

In another aspect, the present invention provides for tonabersat or an analogue of formula I, or a pharmaceutically acceptable composition thereof, for use in the pre-emotive treatment of major cardiovascular disease events, such as myocardial infarction, coronary revascularisation and angina. The patient may be either male or female and most preferably the patient is female.

Accordingly, the present invention additionally provides for tonabersat or an analogue of formula I, or a pharmaceutically acceptable composition thereof, for use in the pre-emptive treatment of major cardiovascular disease events, such as myocardial infarction, coronary revascularisation and angina via administration during the aura phase associated with the potential disease-related event.

In a further aspect, the present invention provides for the use of tonabersat or an analogue of formula I, or a pharmaceutically acceptable composition thereof, in the pre-emptive treatment of epilepsy.

Accordingly, the present invention provides a method for the pre-emptive treatment of epilepsy comprising administering to a patient in need thereof a pharmaceutically effective amount of tonabersat or an analogue of formula I, or a pharmaceutically acceptable composition thereof, during the aura phase associated with a potential epileptic seizure.

In another aspect, the present invention provides for tonabersat or an analogue of formula I, or a pharmaceutically acceptable composition thereof, for use in the pre-emptive treatment of epilepsy.

Accordingly, the present invention additionally provides for tonabersat or an analogue of formula I, or a pharmaceutically acceptable composition thereof, for use in the pre-emptive treatment of epilepsy via administration during the aura phase associated with a potential epileptic seizure.

In a further aspect, the present invention provides for the use of tonabersat or an analogue of formula I, or a pharmaceutically acceptable composition thereof, in the pre-emptive treatment of non-epileptic seizures.

Accordingly, the present invention provides a method for the pre-emptive treatment of non-epileptic seizures comprising administering to a patient in need thereof a pharmaceutically effective amount of tonabersat or an analogue of formula I, or a pharmaceutically acceptable composition thereof, during the aura phase associated with a potential non-epileptic seizure. The patient may be either male or female.

In one embodiment, the present invention provides a method for the pre-emptive treatment of non-epileptic seizures wherein the seizures are either organic or psychogenic seizures.

In a preferred embodiment, the present invention provides a method for the pre-emptive treatment of non-epileptic seizures wherein the seizures are associated with arteriovenous malformation, head injury, drug intoxication, drug toxicity, such as with aminophylline and local anaesthetics, drug withdrawal, infection, such as with meningitis and encephalitis, fever, metabolic disturbances, such as hypoglycaemia, hyponatremia and hypoxia, brain lesions, such as tumours and abscesses, eclampsia, binaural beat brainwave entrainment, haemorrhagic stroke, cerebral venous sinus thrombosis, multiple sclerosis, photophobia, or posttraumatic stress disorder.

In another aspect, the present invention provides for tonabersat or an analogue of formula I, or a pharmaceutically acceptable composition thereof, for use in the pre-emptive treatment of non-epileptic seizures.

Accordingly, the present invention additionally provides for tonabersat or an analogue of formula I, or a pharmaceutically acceptable composition thereof, for use in the pre-emptive treatment of non-epileptic seizures via administration during the aura phase associated with a potential non-epileptic seizure. The patient may be either male or female.

In one embodiment, the present invention provides for tonabersat or an analogue of formula I, or a pharmaceutically acceptable composition thereof, for use in the pre-emptive treatment of non-epileptic seizures wherein the seizures are either organic or psychogenic seizures

In a preferred embodiment, the present invention provides for tonabersat or an analogue of formula I, or a pharmaceutically acceptable composition thereof, for use in the pre-emptive treatment of non-epileptic seizures wherein the seizures are associated with arteriovenous malformation, head injury, drug intoxication, drug toxicity, such as with aminophylline and local anaesthetics, drug withdrawal, infection, such as with meningitis and encephalitis, fever, metabolic disturbances, such as hypoglycaemia, hyponatremia and hypoxia, brain lesions, such as tumours and abscesses, eclampsia, binaural beat brainwave entrainment, haemorrhagic stroke, cerebral venous sinus thrombosis, multiple sclerosis, photophobia, or posttraumatic stress disorder.

The oral compositions of tonabersat or an analogue of formula I, as described in Table 1 have been demonstrated in clinical trials to provide a time to maximum plasma concentration (T_{MAX}) of between 1 and 3 hours with a terminal elimination half life of 24 to 40 hours when administered orally to man. When taken during a migraine attack T_{MAX} may be more variable and prolonged.

For the acute treatment of aura and associated diseases as described herein, it is preferred that the tonabersat or an analogue of formula I composition provides a more rapid onset of action. Compositions comprising tonabersat providing a T_{MAX} of less than 1 hour are preferred.

Accordingly, the present invention provides for a pharmaceutical composition comprising tonabersat or an analogue of formula I, and a pharmaceutically acceptable diluent or carrier, which produces a T_{MAX} of less than 1 hour after administration. Preferably, T_{MAX} is less than 0.9 hours, for example less than 0.8, 0.7, 0.6, 0.5, 0.4, 0.3, 0.2 or 0.1 hours.

In a further aspect, the present invention provides for the use of tonabersator an analogue of formula I, or a pharmaceutically acceptable composition thereof, for the treatment or prevention of aura wherein the composition comprising tonabersat or an analogue of formula I, produces a T_{MAX} of less than 1 hour after administration. Preferably, T_{MAX} is less than 0.9 hours, for example less than 0.8, 0.7, 0.6, 0.5, 0.4, 0.3, 0.2 or 0.1 hours after administration. The aura may, for example, be associated with migraine, epilepsy, non-epileptic seizures, stroke, or major cardiovascular disease events, such as myocardial infarction, coronary revascularisation or angina. In one preferred embodiment the treatment is acute. In another preferred embodiment the treatment is prophylactic.

Accordingly, the present invention provides a method for the treatment or prevention of aura comprising administering to a patient in need thereof a pharmaceutically effective amount of tonabersat or an analogue of formula I, or a pharmaceutically acceptable composition thereof, wherein the composition comprising tonabersat produces a T_{MAX} of less than 1 hour after administration. Preferably, T_{MAX} is less than 0.9 hours, for example less than 0.8, 0.7, 0.6, 0.5, 0.4, 0.3, 0.2 or 0.1 hours after administration.

In another aspect, the present invention provides for tonabersat or an analogue of formula I, or a pharmaceutically acceptable composition thereof, for use in the treatment or prevention of aura wherein the composition comprising tonabersat or an analogue of formula I, produces a T_{MAX} of less than 1 hour after administration. Preferably, T_{MAX} is less than 0.9 hours, for example less than 0.8, 0.7, 0.6, 0.5, 0.4, 0.3, 0.2 or 0.1 hours after administration.

For the avoidance of doubt, the compositions having a more rapid onset of action, i.e. reduced T_{MAX}, as described herein are considered suitable for the treatment or prevention of all diseases referred to herein, including the premonitory symptoms of migraine, pre-emptive treatment of migraine with or without aura, migraine recurrence, epilepsy, non-epileptic seizures, stroke, or major cardiovascular disease events, such as myocardial infarction, coronary revascularisation and angina. The compositions may be used for both acute and prophylactic treatment.

Also included within the scope of the present invention are polymorphs, solvates and radiolabelled derivatives of tonabersat or an analogue of formula I, and pharmaceutically acceptable compositions thereof. References to tonabersat or an analogue of formula I, include such polymorphs, solvates and radiolabelled derivatives thereof.

Tonabersat or an analogue of formula I, may be delivered alone, but will generally be delivered in the form of a pharmaceutically acceptable composition thereof, which comprises tonabersat and one or more pharmaceutically acceptable diluents or carriers selected with regard to the intended route of administration.

Treatment with tonabersat or an analogue of formula I, or a pharmaceutically acceptable composition thereof, may be conducted at a unit dose of between 1 to 1000 mg, suitably 1 to 500 mg, for example an amount in the range of from 2 to 400 mg such as 2, 5, 10, 20, 30, 40, 50, 80, 100, 200, 300 and 400 mg of the active compound.

Unit doses will normally be administered once or more than once per day, for example 1, 2, 3, 4, 5 or 6 times a day, more usually 1 to 4 times a day, such that the total daily dose is normally in the range, for a 70 kg adult of 1 to 1000 mg, for example 1 to 500 mg, that is in the range of approximately 0.01 to 15 mg/kg/day, more usually 0.1 to 6 mg/kg/day, for example 1 to 6 mg/kg/day.

Preferably, the tonabersat or an analogue of formula I, or a pharmaceutically acceptable salt thereof, is administered to the patient at dose ranges of approximately 0.01 to 15 mg/kg/day, more usually 0.1 to 6 mg/kg/day, for example 1 to 6 mg/kg/day.

It is preferred that the compound of formula (I) is administered in the form of a pharmaceutical composition, such as a composition for oral, including sub-lingual, intranasal, rectal, topical, parenteral (especially intravenous), or ocular administration.

Pharmaceutical compositions suitable for the delivery of tonabersat or an analogue of formula I, and methods for their preparation will be readily apparent to those skilled in the art. Such compositions and methods for their preparation may be found, for example, in Remington's Pharmaceutical Sciences, 19th Edition (Mack Publishing Company, 1995).

Compositions suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations. Liquid formulations include suspensions, solutions, syrups and elixirs. Liquid formulations may also be prepared by the reconstitution of a solid, for example, from a sachet.

Compositions for oral administration may be formulated to be immediate and/or modified release. Modified release formulations include delayed-, sustained-, pulsed-, controlled-, targeted and programmed release.

Compositions suitable for parenteral administration include injectable and infusible aqueous or oily blends, mixtures, suspensions, solutions, emulsions and low-viscosity gel preparations. Compositions for parenteral administration may be formulated to be immediate and/or modified release. Modified release formulations include delayed-, sustained-, pulsed-, controlled-, targeted and programmed release.

Tonabersat or an analogue of formula I, may also be administered intranasally or by inhalation, typically in the form of a dry powder from a dry powder inhaler, or as an aerosol spray.

Tonabersat or an analogue of formula I, may also be administered rectally or vaginally, for example, in the form of a suppository, pessary, or enema.

The tonabersat or an analogue of formula I, compositions may also be in the form of fast-dispersing dosage forms such as those described in Expert Opinion in Therapeutic Patents, 11 (6), 981-986, by Liang and Chen (2001) and Verma R K et. al. Current Status of Drug Delivery Technologies and Future Directions, Pharmaceutical Technology On-Line, 2001, 25(2), 1-14. Such dosage forms are also known as oral fast-dissolving, rapid-dissolve, rapid-melt, mouth-dissolving and fast-disintegrating tablet. The composition may be in solid form which melts on contact with the tongue of the patient, for example in the form of disintegrating tablets sold under the trade name ZYDIS® (RP Scherer, U K). Alternatively, the composition may be in the form of the EFVDAS (effervescent drug absorption system, Elan Corporation), Fast Melt (highly porous microfine matrix tablet, Elan Corporation), Flashdose (floss matrix utilising shearform technology, (Fuisz Technologies, USA), Flashtab (orodispersible multiparticulate tablet, Prographarm, France), Multiflash (fast disintegrating multi-unit, multiparticulate tablet, Prographarm), Orasolv (effervescent dispersed microcapsule tablet, Cima Labs Inc, USA), Wowtab tablets (Yamanouchi Pharma Technologies, USA), LYOC (freeze dried fast dispersing tablets, Farmalyoc, France) or Quicksolve (freeze dried fast dispersing tablets, Janssen Pharamceutica, USA).

Other suitable formulation technologies may include INDAS (insoluble drug absorption system, Elan Corporation), which utilises a stabilised amorphous form of the drug with enhanced solubility, NanoCrystal technology (Elan Corporation), which utilises nanoparticles of the drug, typically having a particle size of less than 400 nm in diameter, or SoftGel (RP Scherer), which utilises a soft gelatin capsule formulation.

Alternatively, compositions comprising co-crystals (Chemical & Engineering News, 2007, 85(25), 17-30) of tonabersat may be utilised thereby enhancing the rate of dissolution rate and rate of absorption of the drug. Such co-crystals may be formed by slow evaporation and/or sonication of solutions comprising equimolar or stoichiometric concentrations of tonabersat and co-host. Suitable solvents for the production of co-crystals of tonabersat comprise acetone, THF, ethyl acetate, methanol, ethanol, isopropyl alcohol, chloroform or mixtures thereof. Suitable mixtures may include, for example, 1:1 mixtures of methanol and chloroform or ethanol and THF or mixtures of ethanol with heptane. Suitable co-hosts may include glutaric acid or citric acid. Preferred solvents for glutaric acid and citric acid include acetone, ethanol and 1:1 mixture of chloroform and methanol.

Accordingly, the present invention additionally provides co-crystals of tonabersat comprising glutaric acid or citric acid. A preferred co-crystal comprises tonabersat and glutaric acid.

Additionally, the present invention provides pharmaceutical compositions comprising co-crystals of tonabersat comprising glutaric acid or citric acid. A preferred pharmaceutical composition comprises co-crystals comprising tonabersat and glutaric acid.

The formulation technologies described herein may advantageously provide more rapid drug dissolution and absorption. For those compositions that disintegrate in the oral cavity, such as beneath the tongue, the rate of absorption may be increased and first-pass metabolism effects reduced.

The following are given by way of example only to illustrate and aid understanding of the invention:

Studies with tonabersat have employed a number of different formulations including:
Direct compression tablets 0.05, 1.0, 10 and 25 mg with tablet core weight 250 mg
Direct compression tablets 15, 25, 40 and 80 mg with tablet core weight 400 mg
Direct compression tablets 20 mg with core weight 400 mg
Nanoparticulate tablets 10, 20 and 40 mg with tablet core weight 400 mg

The direct compression tablets utilise micronized drug substance whilst the nanoparticulate tablets were direct compression tablets utilising wet bed milled spray dried nanoparticulate drug substance. Clinical trials have been conducted utilising 10, 20, 30, 40, 60 and 80 mg round white uncoated direct compression tablets with a core weight of 400 mg with the following unit composition (20 mg tablet only presented; all other strengths differ only in tonabersat and lactose content):

A representative formulation suitable for use in the present invention is detailed in Table 1.

TABLE 1
Unit composition of tonabersat 20 mg

| | 20 mg Tablet |
|---|---|
| Ingredient | Quantity (mg) |
| Tonabersat | 20.0 |
| Lactose | 330.0 |
| Microcrystalline Cellulose | 20.0 |
| Sodium Starch Glycollate, | 24.0 |
| Type A | |
| Colloidal Silicon Dioxide | 2.0 |
| Magnesium Stearate | 4.0 |
| Total Weight | 400.0 |

Pharmacological Data

A single centre, double-blind, randomised, placebo controlled crossover study to evaluate the efficacy and tolerability of tonabersat in the prophylaxis of migraine in patients presenting with migraine with aura was conducted.

Study Duration:

Twelve week treatment period followed by a 4 week wash-out period before cross-over to 12 weeks treatment with the alternative trial medication.

Objectives:

To investigate the efficacy of tonabersat compared to placebo in the reduction of the number of aura attacks and the number of migraine headache days in patients with migraine with aura.

Methodology:

Following screening, patients were randomised to active or placebo treatment for 12 weeks. During the first two weeks of the treatment period, patients received treatment with tonabersat 20 mg (one tablet daily) or matching placebo. After two weeks, the dose was increased to tonabersat 40 mg per day (two tablets taken once daily [od]) with a similar increase in the number of placebo tablets taken. Depending on tolerability during the treatment period, the dose may have been reduced to 20 mg per day (or one placebo tablet) for the remainder of the treatment period. On completion of the first treatment period patients received placebo tablets (one tablet daily) for 4 weeks before starting the second treatment period when the alternative trial medication was administered. The design of the second period was the same as the first.

Diagnosis and Main Criteria for Inclusion:

Diagnosis: Migraine with aura meeting the diagnostic criteria of the International Classification of Headache Disorders (Edition 2).

Main criteria for inclusion: Male or female patients between 18-65 years of age with an established history of migraine of at least one year with aura meeting the diagnostic criteria of the International Classification of Headache Disorders (Edition 2).

Test product, dose and mode of administration:

Tonabersat 20 mg tablets: total daily dose 20 or 40 mg orally (po) od.

Reference therapy, dose, and mode of administration:

Placebo tablets matching the appearance of tonabersat tablets: one or two tablets taken po od.

Primary Efficacy Variables were mean number of aura attacks and mean number of migraine headache days experienced in each treatment period.

The outcome data from the clinical study in 31 patients presenting with migraine with aura showed that patients treated with tonabersat had a statistically significant (p=0.01) 50% reduction in aura attacks and a 37.5% reduction (p=0.08) in migraine headache days compared to patients treated with placebo.

1-11. (canceled)

12. A method for the treatment of one or more of the premonitory symptoms of migraine, the pre-emptive treatment of migraine or the prevention of migraine recurrence comprising administering to a patient in need thereof a pharmaceutically effective amount of tonabersat, or an analogue or formula 1 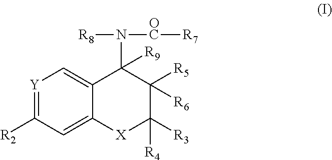
Y is C—R₁;
R₁ is acetyl;
R₂ is hydrogen, C₃₋₈ cycloalkyl, C₁₋₆ alkyl optionally interrupted by oxygen or substituted by hydroxy, C₁₋₆ alkoxy or substituted aminocarbonyl, C₁₋₆alkylcarbonyl, C₁₋₆ alkoxycarbonyl, C₁₋₆ alkylcarbonyloxy, C₁₋₆ alkoxy, nitro, cyano, halo, trifluoromethyl, or CF₃S; or a group CF₃-A-, where A is —CF₂—,
—CO—, —CH₂—, CH(OH), SO₂, SO, CH₂—O, or CONH; or a group CF₂H-A′- where A′ is oxygen, sulphur, SO, SO₂, CF₂ or CFH; trifluoromethoxy, C₁₋₆ alkylsulphinyl, perfluoro C₂₋₆ alkylsulphonyl, C₁₋₆ alkylsulphonyl, C₁₋₆ alkoxysulphinyl, C₁₋₆ alkoxysulphonyl, aryl, heteroaryl, arylcarbonyl, heteroarylcarbonyl, phosphono, arylcarbonyloxy, heteroarylcarbonyloxy, arylsulphinyl, heteroarylsulphinyl, arylsulphonyl, or heteroarylsulphonyl in which any aromatic moiety is optionally substituted, C₁₋₆ alkylcarbonylamino, C₁₋₆ alkoxycarbonylamino, C₁₋₆ alkyl-thiocarbonyl, C₁₋₆ alkoxy-thiocarbonyl, C₁₋₆ alkyl-thiocarbonyloxy, 1-mercapto C₂₋₇ alkyl, formyl, or aminosulphinyl, aminosulphonyl or aminocarbonyl, in which any amino moiety is optionally substituted by one or two C₁₋₆ alkyl groups, or C₁₋₆ alkylsulphinylamino, C₁₋₆ alkylsulphonylamino, C₁₋₆ alkoxysulphinylamino or C₁₋₆ alkoxysulphonylamino, or ethylenyl terminally substituted by C₁₋₆ alkylcarbonyl, nitro or cyano, or —C(C₁₋₆ alkyl)NOH or —C(C₁₋₆ alkyl)NNH₂ ; or amino optionally substituted by one or two C₁₋₆ alkyl or by C₂₋₇ alkanoyl; one of R₃ and R₄ is hydrogen or C₁₋₄ alkyl and the other is C₁₋₄ alkyl, CF₃ or CH₂X^{a} is fluoro, chloro, bromo, iodo, C₁₋₄ alkoxy, hydroxy, C₁₋₄ alkylcarbonyloxy, —S—C₁₋₄ alkyl, nitro, amino optionally substituted by one or two C₁₋₄ alkyl groups, cyano or C₁₋₄ alkoxycarbonyl; or R₃ and R₄ together are C₂₋₅ polymethylene optionally substituted by C₁₋₄ alkyl;
R₅ is C₁₋₆ alkylcarbonyloxy, benzoyloxy, ONO₂, benzyloxy, phenyloxy or C₁₋₆ alkoxy and R₆ and R₉ are hydrogen or R₅ is hydroxy and R₆ is hydrogen or C₁₋₂ alkyl and R₉ is hydrogen;
R₇ is heteroaryl or phenyl, both of which are optionally substituted one or more times independently with a group or atom selected from chloro, fluoro, bromo, iodo, nitro, amino optionally substituted once or twice by C₁₋₄ alkyl, cyano, azido, C₁₋₄ alkoxy, trifluoromethoxy and trifluoromethyl;
R₈ is hydrogen, C₁₋₆ alkyl, OR₁₁ or NHCOR₁₀ wherein R₁₁ is hydrogen, C₁₋₆ alkyl, formyl, C₁₋₆ alkanoyl, aroyl or aryl-C₁₋₆ alkyl and R₁₀ is hydrogen, C₁₋₆ alkyl, C₁₋₆ alkoxy, mono or di C.sub.1-6 alkyl amino, amino, amino-C.sub.1-6 alkyl, hydroxy-C₁₋₆ alkyl, halo-C₁₋₆ alkyl, C₁₋₆ acyloxy-C₁₋₆ alkyl, C₁₋₆ alkoxycarbonyl-C₁₋₆-alkyl, aryl or heteroaryl; the R₈—N—CO—R₇ group being cis to the R₅ group;
and X is oxygen or NR₁₂ where R₁₂ is hydrogen or C₁₋₆ alkyl.or a pharmaceutically acceptable composition thereof.

13. (canceled)

14. A method according to claim 12, wherein the premonitory symptoms are selected from the group consisting of irritability, euphoria, elation, physical hyperactivity, fatigue, excessive yawning, excessive sleepiness, rhinitis, chronic rhinitis, sinusitis, increased sensitivity to light and sound, unusual hunger, craving for certain foods, depression, mental withdrawal, behaviour sluggishness, feeling tired, poor concentration, muscle weakness, anorexia and fluid retention.

15. A method according to claim 12, wherein the tonabersat or an analogue of formula 1, is administered during the premonitory symptom phase associated with a migraine attack.

16-18. (canceled)

19. A method for the treatment or prevention of aura comprising administering to a patient in need thereof a pharmaceutically effective amount of tonabersat or an analogue or formula 1, or a pharmaceutically acceptable composition thereof.

20. A method according to claim 19, wherein the aura is in a patient with either a history of, or at higher risk of suffering from, migraine with or without aura, epilepsy, non-epileptic seizures, stroke or cardiovascular disease, including major cardiovascular disease events, such as myocardial infarction, coronary revascularisation or angina.

21-25. (canceled)

26. A method for the pre-emptive treatment of stroke, major cardiovascular disease events, including myocardial infarction, coronary revascularisation and angina, epilepsy or non-epileptic seizures comprising administering to a patient in need thereof a pharmaceutically effective amount of tonabersat or an analogue or formula 1, or a pharmaceutically acceptable composition thereof.

27. A method according to claim 26 wherein the tonabersat, or composition thereof, is administered during the aura phase associated with a potential stroke, potential epileptic or non-epileptic seizure.

28-32. (canceled)

33. A method according to claim 27, wherein the non-epileptic seizures are either organic or psychogenic seizures.

34. (canceled)

35. A method for the treatment or prevention of aura according to claim 19, comprising administering to a patient in need thereof a pharmaceutically effective amount of tonabersat or an analogue or formula 1, or a pharmaceutically acceptable composition thereof, wherein the composition comprising tonabersat produces a T_{MAX} of less than 1 hour after administration.

36. A method according to claim 19, wherein the aura is in a patient with a history of migraine with aura.

37. A method according to claim 19, wherein the tonabersat, or composition thereof, is administered during the premonitory symptom phase associated with a migraine attack.

38. A method according to claim 19, wherein the aura is in a patient with a history of epilepsy or non-epileptic fits.

39. A method according to claim 19 wherein the aura is in a patient with a history of, or at higher risk of suffering from, a stroke, major cardiovascular disease events, such as myocardial infarction, coronary revascularisation or angina.

40. A method according to claim 19, wherein the aura is in a patient with a history of, or at higher risk of suffering from, a stroke.

41. A method according to claim 33, wherein the seizures are associated with arteriovenous malformation, head injury, drug intoxication, drug toxicity, such as with aminophylline and local anaesthetics, drug withdrawal, infection, such as with meningitis and encephalitis, fever, metabolic disturbances, such as hypoglycaemia, hyponatremia and hypoxia, brain lesions, such as tumours and abscesses, eclampsia, binaural beat brainwave entrainment, haemorrhagic stroke, cerebral venous sinus thrombosis, multiple sclerosis, photophobia, or posttraumatic stress disorder.
